(19) United States
(12) Patent Application Publication
(10) Pub. No.: US 2006/0094068 A1
(43) Pub. Date: May 4, 2006

(54) Predictive markers in cancer therapy

(76) Inventors: Sarah S. Bacus, Tuscon, AZ (US); Myra R. Herrle, Durham, NC (US); L. Edward Kirk, Durham, NC (US); Neil L. Spector, Durham, NC (US); Michael T. Stocum, Durham, NC (US); Wenle Xia, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/529,922

(22) PCT Filed: Apr. 24, 2003
(86) PCT No.: PCT/US03/12739

§ 371 Date: Mar. 30, 2005

Related U.S. Application Data
(60) Provisional application No. 60/389,795, filed on Jun. 19, 2002.
(60) Provisional application No. 60/432,811, filed on Dec. 11, 2002.
(60) Provisional application No. 60/432,943, filed on Dec. 11, 2002.
(60) Provisional application No. 60/451,978, filed on Mar. 5, 2003.

(51) Int. Cl. G01N 33/574; A61K 31/52

(52) U.S. Cl. 435/7.23; 514/263.1

(57) ABSTRACT

Molecular markers useful in medicine response tests are provided, as an aid in determining whether an individual subject s tumor is responding to treatment with EGF and/or erbB2 inhibitors. Markers include phosphorylated ERK protein

### FIELD OF THE INVENTION

### Background

The ErbB Family

The erbB family of type I receptor tyrosine kinases includes erbB1 (also known as the epidermal growth factor receptor (EGFR or HER1), erbB2 (also known as Her2), erbB3, and erbB4. These receptor tyrosine kinases are widely expressed in epithelial, mesenchymal, and neuronal tissues where they play a role in regulating cell proliferation, survival, and differentiation (Sibilia and Wagner, Science, 269: 234 (1995); Threadgill et al., Science, 269: 230 (1995)). Overexpression of wild-type erbB2 or EGFR, or expression of constitutively activated receptor mutants, transforms cells in vitro (Di Fiore et al., 1987; DiMarco et al, Oncogene, 4: 831 (1989); Hudziak et al., Proc. Natl. Acad. Sci. USA., 84:7159 (1987); Qian et al., Oncogene, 10:211 (1995)). Overexpression of erbB2 or EGFR has been correlated with a poorer clinical outcome in some breast cancers and a variety of other malignancies (Slamon et al., Science, 235: 177 (1987); Slamon et al., Science, 244:707 (1989); Bacus et al, Am. J. Clin. Path., 102:S13 (1994)).

A family of peptide ligands regulates erbB receptor signaling, and includes epidermal growth factor (EGF) and transforming growth factor a (TGF-α), each of which binds to EGFR (Reise and Stern, Bioessays, 20:41 (1998); Salomon et al., Crit. Rev. Oncol. Hematol., 19: 183 (1995)). Ligand binding induces erbB receptor homo- and heterodimerization, which in turn leads to receptor autophosphorylation and activation. ErbB2 is the preferred heterodimeric partner for EGFR, erbB3, and erbB4 (Graus-Porta et al., EMBO J., 16:1647 (1997); Tzahar et al., Mol. Cell. Biol., 16: 5276 (1996)). A number of soluble ligands have been identified for EGFR, erbB3, and erbB4, but none have been identified for erbB2, which seems to be transactivated following heterodimerization (Ullrich and Schlessinger, Cell, 61: 203 (1990); Wada et al., Cell, 61: 1339 (1990); Karunagaran et al., EMBO J., 15:254 (1996); Stem and Kamps, EMBO J., 7: 995 (1988)).

With the exception of erbB3, all erbB receptor family members share a highly conserved cytoplasmic tyrosine kinase domain. Autophosphorylation of specific cytoplasmic tyrosine residues establishes binding sites for Src-homology 2 (SH2) and phosphotyrosine-binding-domain containing proteins that in turn link to downstream effectors involved in cell proliferation (mitogen-activated protein kinases or MAPK; also known as Erk1/2) and survival (phosphatidylinositol 3-kinase/AKT) pathways (Olayioye et al., Mol. Cell. Biol., 18:5042 (1998); Luttrell et al., Proc. Natl. Acad. Sci. USA. 91:83 (1994); Levkowitz et al., Oncogene, 12:1117 (1996); Klapper et al., Adv. Cancer Res., 77:25 (2000); Egan and Weinberg, Nature, 365:781 (1993); Kavanaugh and Williams, Science, 266: 1862(1994); Daly R J. Growth Factors, 16:255 (1999)).

The significance of EGFR or erbB2 receptor overexpression in tumor physiology has been investigated. Additionally, increased expression of the ligands EGF or TGF-α has been reported as a poor prognostic indicator in some cancer patients (Grandis et al., J. Natl. Cancer Inst., 90:824 (1998); Albanell et al, Cancer Res., 61: 6500 (2001)), and locally increased concentrations of EGF or other ligands in the tumor microenvironment appear to be capable of maintaining heterodimers in an activated state even in the absence of receptor overexpression (Albanell et al, Cancer Res., 61: 6500 (2001); DiMarco et al, Oncogene, 4: 831 (1989); Howell et al., J. Biol. Chem., 273:9214 (1998); Jiang et al., J. Biol. Chem., 273:31471 (1998)).

Trastuzumab (Herceptin™), a humanized anti-erbB2 monoclonal antibody has been approved for the treatment of breast cancers that either overexpress erbB2, or that demonstrate erbB2 gene amplification (Cobleigh et al, J. Clin. Oncol., 17:2639 (1999)). Similarly, several anti-EGFR targeted approaches are currently undergoing clinical investigation, including C225, a human-mouse chimeric anti-EGFR mAb (Goldstein et al., Clin. Cancer Res., 1:1311 (1995); Levitzki and Gazit, Science, 267:1782 (1995); Mendelsohn, Clin. Cancer Res., 3:2703 (1997)) and ZD1839 (Iressa™, a small molecule compound; see Ranson et al., Exp. Rev. Anticancer Ther. 2:161(2002)).

Because heterodimers of erbB2 and EGFR can elicit potent mitogenic signals, interrupting both erbB2 and EGFR simultaneously is a potential therapeutic strategy (Earp et al., Breast Cancer Res. Treat., 35:115 (1995)). Small molecule, dual EGFR-erbB2 tyrosine kinase inhibitors have been identified and their pre-clinical anti-tumor activities reported (Fry et al., Proc. Natl. Acad. Sci. USA., 95:12022 (1998); Cockerill et al., Bioorganic Med. Chem. Letts., 11:1401 (2001); Rusnak et al., Cancer Res., 61:7196 (2001); Rusnak et al., Mol. Cancer Therap., 1:85 (2001)).

Recently, the combination of Herceptin™ (an anti-erbB2 monoclonal antibody) and C225 (an anti-EGFR Mab) was shown to exhibit enhanced growth inhibition in OVCA 420 human ovarian carcinoma cells compared with either mAb alone (Ye et al., Oncogene, 18:731 (1999)). However, EGF and C225 have comparable binding affinities for EGFR, and EGF was able to reverse the growth inhibitory effects of combined Herceptin™ and C225. This combined approach may therefore be problematic in the clinic, where patients may have increased levels of EGF receptor ligands (Ye et al., Oncogene, 18:731 (1999)).

Due to the network of growth factor receptors, ligands, and downstream cell proliferation and cell survival effector molecules, inhibiting specific receptor tyrosine kinases may not be an effective therapeutic strategy in all individuals with cancer, as various compensatory pathways may exist to overcome the therapeutic inhibition. Accordingly, it will be useful to identify biological markers that indicate, in an individual subject, whether the subject's tumor is responding to a particular therapeutic intervention. While tumor size or progression of disease has traditionally been used to determine whether an individual was responding to a particular therapy, use of molecular markers may allow earlier identification of responders and non-responders. Non-responders can be offered alternate therapy, and spared potential side effects of a therapy that is ineffective for their specific tumor.

It would be useful to identify one or a combination of molecular markers capable of indicating whether an individual's tumor is responding to treatment with EGF and/or erbB2 inhibitors. Such markers would help (i) identify in which clinical settings and patient populations the therapeutic approach is most likely to be effective, and (ii) assess, in individual patients, whether the patient's tumor is responding to a specific treatment.

### BRIEF DESCRIPTION OF THE FIGURES

FIG. 1. Inhibition of activated erbB2 receptor and ERK1/2 MAP kinases by GW572016 in an erbB2 overexpressing mammary epithelial cell line. Activated erbB2 (p-Tyr/erbB2), activated Erk1/2 (p-Erk1/2), and total Erk1/2 were assessed by Western blot in S1 cells treated with GW572016 at the indicated concentrations (0.5-5.0 μM) for 72 h. Controls were treated with vehicle alone (V, DMSO at a final concentration of 0.1%).

FIG. 2a. The effects of EGF and GW572016 on the activation state of erbB2 and downstream Erk1/2 and AKT in BT474 (erbB2 overexpressing) tumor cell lines. Cells were cultured in the presence or absence of GW572016 (1 μM) in serum-free medium for 24 hours. EGF (50 ng/ml) was added to cell cultures as indicated. Equal amounts of protein were used to assess activated erbB2 (p-Tyr/erbB2) in BT474 cells, and Erk1/2, activated ERK1/2 (p-Erk1/2), AKT, and activated AKT (p-AKT) by Western blot.

FIG. 2b. The effects of EGF and GW572016 on the activation state of EGFR and downstream Erk1/2 and AKT in HN5 (EGFR overexpressing) tumor cell lines. Cells were cultured in the presence or absence of GW572016 (5 μM) in serum-free medium for 24 hours. EGF (50 ng/ml) was added to cell cultures as indicated. Equal amounts of protein were used to assess activated EGFR (p-Tyr/EGFR) and Erk1/2, p-Erk1/2, AKT, p-AKT by Western blot.

FIG. 3 graphs GW572016-induced apoptosis of S1 cells, an erbB2 overexpressing mammary epithelial cell line. The percentage of cells in G1, S phase, and G2/M are indicated. The sub-G1 peak represents the apoptotic fraction. FIG. 3a: untreated control cells. FIG. 3b: cells treated with vehicle (0.1% DMSO). FIG. 3c: cells treated with GW572016 (51μM).

FIG. 4. Comparison by Western Blot of the effects of GW572016 with Herceptin™ on activated Erk1/2 in BT474 (erbB2 overexpressing) and HN5 (EGFR over-expressing) cell lines.

FIG. 5 compares the effects of GW572016 and Herceptin™ on the activation state of erbB2, EGFR and downstream Erk1/2 in Hb4a cells (cells expressing low levels of both erbB2 and EGFR). Addition of EGF increased p-Tyr/EGFR (compare lanes 1 and 2). Addition of GW572016 decreased baseline p-Tyr/EGFR, p-erk1/2, and p-Tyr/ErbB2 levels (compare lanes 1 and 3); GW572016 also blocked EGF-stimulated increases of p-Tyr/EGFR (compare lanes 2 and 4).

FIG. 6a illustrates GW572016 inhibition of activated EGFR in HN5 (EGFR overexpressing) xenografts. Animals were treated with Vehicle (control) or GW572016 at 10 mg/kg, 30 mg/kg or 100 mg/kg. Each treatment group consisted of three animals (indicated as 1, 2 and 3); each animal was biopsied at the same tumor implant before (Pre) and after (Post) the final dose.

FIG. 6b illustrates GW572016 inhibition of activated Erk1/2 and AKT in HN5 (EGFR overexpressing) xenografts. Three animals treated with 30 mg/kg GW572016were assessed (indicated as 1, 2 and 3); each animal was biopsied at the same tumor implant before (Pre) and after (Post) the final dose. Total Erk1/2, total AKT, activated Erk1/2 (p-Erk1/2), and activated AKT (p-AKT) were assessed by Western blot loading equal amounts of protein from tumor biopsies.

FIG. 7 illustrates GW572016 inhibition of ErbB-2 and downstream Erk1/2 activation in BT474 (erbB2 overexpressing) xenografts. Animals were treated with GW572016 (100 mg/kg) or vehicle control; each treatment group consisted of three animals (vehicle=lanes 1, 2 and 3; GW572016=lanes 4, 5 and 6). The tumor implant was removed after the final treatment dose. Activated receptor (p-Tyr/ErbB-2) was assessed by IP Western blot and total ErbB-2 steady state protein (ErbB-2), total Erk1/2 and activated Erk1/2 (p-Erk1/2) were assessed by Western blot loading equal amounts of protein from tumor biopsies. Treatment with GW572016 decreased activated p-Tyr/ErbB2 and p-Erk1/2.

### SUMMARY

A first aspect of the present invention is a method of assessing, in a human subject needing treatment for an EGFR-expressing solid tumor, whether the subject is likely to exhibit a favorable clinical response to such treatment. The method comprises determining the pre-treatment level of pERK in the tumor, administering a therapeutically effective amount of an EGFR inhibitor, an erbB2 inhibitor, or a dual EGFR/erbB2 inhibitor, and determining the level of pERK in the tumor after an initial period of treatment with the therapeutic agent. A decrease in the pERK level indicates that the subject is more likely to exhibit a favorable clinical response to the treatment, compared to a subject with no change or an increase in pERK levels.

A further aspect of the present invention is a method of assessing, in a human subject in need of treatment for an erbB2-expressing solid tumor, whether the subject is likely to exhibit a favorable clinical response to such treatment. The method comprises

determining the pre-treatment level of pERK in the tumor, administering a therapeutically effective amount of an EGFR inhibitor, an erbB2 inhibitor, or a dual EGFR/erbB2 inhibitor, and determining the level of pERK in the tumor after an initial period of treatment with said therapeutic agent, where a decrease in the pERK level indicates that the subject is more likely to exhibit a favorable clinical response to the treatment, compared to a subject with no change or an increase in pERK levels.

### DETAILED DESCRIPTION

Attention has focused on developing therapeutically active monoclonal antibodies (mAb) or small molecule kinase inhibitors that target either EGFR or erbB2, for the treatment of cancer. Additionally, as increased concentrations of EGF or other ligands appear to be capable of maintaining heterodimers in an activated state even in the absence of receptor overexpression, it is important to develop therapeutic strategies that are not dependent upon receptor overexpression for anti-tumor activity.

A number of small molecule, dual EGFR-erbB2 tyrosine kinase inhibitors have been identified and their pre-clinical anti-tumor activities reported (Fry et al., Proc. Natl. Acad. Sci. USA., 95:12022 (1998); Cockerill et al., Bioorganic Med. Chem. Letts., 11:1401 (2001); Rusnak et al., Cancer Res., 61:7196 (2001); Rusnak et al., Mol. Cancer Therap., 1:85 (2001)). GW572016 is a potent reversible, dual inhibitor of the tyrosine kinase domains of both EGFR and erbB2, with IC₅₀ values against purified EGFR and erbB2 of 10.2 and 9.8 nM, respectively (Rusnak et al., Mol. Cancer Therap., 1:85 (2001)). Recent reports have demonstrated that GW572016 inhibits EGFR or erbB2 autophosphorylation in tumor cell lines that overexpress either receptor (Rusnak et al., Mol. Cancer Therap., 1:85 (2001)), an effect that was primarily associated with tumor cell growth arrest. The chemical name of GW572016 is N-{3-chloro-4-[(3-fluorobenzyl)oxy]phenyl}-6-[5-({[2-methylsulfonyl)ethyl]amino}methyl)-2-furyl]-4-quinazolinamine (WO 99 35146, Carter et al.); a ditosylate form is disclosed in WO 02 02552 (McClure et al).

As reported herein GW572016 inhibits not only baseline activation of both erbB2 and EGFR receptors, but also interrupts downstream activation of Erk1/2 MAP kinases and AKT. GW572016 was shown to inhibit signal transduction in EGF-stimulated tumor lines that did not overexpress EGFR, and exogenous EGF did not reverse the anti-tumor effects of GW572016.

The studies reported herein examine the in vivo effects of a dual EGFR/erbB2 inhibitor in the same tumor biopsied both before and after treatment. This approach was taken in an attempt to minimize inter-subject variability in baseline expression of activated EGFR and erbB2. GW572016 inhibits tumor xenograft growth (Rusnak et al., Mol. Cancer Therap., 1:85 (2001)); the present studies clarify its mechanism of action by demonstrating that GW572016 inhibits the activation of proliferation and survival pathways in both erbB2 and EGFR-dependent tumors. Additionally, the clinical response of human cancer patients after eight weeks of treatment with GW572016 was determined to be correlated with changes in levels of pERK, pAKT, and cyclin D1 in sequential tumor biopsies.

As used herein, a method of screening or assessing a subject as an aid in predicting the subject's response to a therapeutic treatment (a ‘medicine response prognosis’) should not be confused with the use of disease prognosis markers. Certain molecular markers are known as indicators of more aggressive cancers and are associated with decreased average survival time (compared to subjects whose tumors do not express such markers). The present invention is not directed to general disease prognosis markers, but to the use of specified biological markers to assess an individual's response to a therapeutic treatment.

Methods of the present invention are directed to the use of biomarkers to monitor a subject's response to a therapeutic treatment, to determine whether the subject is likely to have a favorable clinical response to that treatment. More specifically, methods of the present invention are directed to monitoring changes in levels of biomarkers in the early period of therapeutic treatment of a solid tumor with an erbB2 inhibitor, an EGFR inhibitor, or a dual erbB2/EGFR inhibitor, to identify subjects who are likely to exhibit a favorable clinical response to such treatment (compared to the likelihood of such a response in the general population).

Methods of the present invention are further directed to the use of levels of biomarkers prior to initiating therapy, as an aid in predicting whether the subject will have a favorable clinical response to a specified therapeutic treatment. More specifically, methods of the present invention are directed to determining levels of biomarkers prior to therapeutic treatment of a solid tumor with an erbB2 inhibitor, an EGFR inhibitor, or a dual erbB2/EGFR inhibitor, to identify subjects who are likely to respond favorably (clinically) to such treatment (compared to the likelihood of such a response in the general population). As used herein, predictive is not meant to imply a 100% predictive ability, but to indicate that subjects with certain characteristics are more likely to experience a favorable clinical response than subjects who lack such characteristics. However, as will be apparent to one skilled in the art, some individuals identified as more likely to experience a favorable clinical response will nonetheless experience progression of disease. It will further be apparent to one skilled in the art that, just as certain conditions are identified herein as associated with an increased likelihood of a favorable clinical response, the absence of such conditions will be associated with a decreased likelihood of a favorable clinical response.

As used herein, a subject refers to a mammal, including humans, canines and felines. Preferably subjects treated with the present methods are humans.

As used herein, a ‘favorable response’ (or ‘favorable clinical response’) to a treatment refers to a biological or physical response that is recognized by those skilled in the art as indicating a decreased rate of tumor growth, compared to tumor growth that would occur in the absence of any treatment. “Favorable clinical response” as used herein is not meant to indicate a cure. A favorable clinical response to therapy may include a lessening of symptoms experienced by the subject, an increase in the expected or achieved survival time, a decreased rate of tumor growth, cessation of tumor growth (stable disease), and/or regression of the tumor mass (each as compared to that which would occur in the absence of therapy).

As is well known in the art, tumors are frequently metastatic, in that a first (primary) locus of tumor growth spreads to one or more anatomically separate sites. As used herein, reference to “a tumor” in a subject includes not only the primary tumor, but metastatic tumor growth as well. In some cases, the primary tumor may be surgically inaccessible while metastases are more readily accessible.

As used herein, an erbB2 inhibitor is an agent that inhibits or reduces the formation of p-Tyr/erbB2 (activated erbB2), compared to the formation of p-Tyr/erbB2 that would occur in the absence of the erbB2 inhibitor. Such inhibitors include small chemical molecules and biologic agents such as monoclonal antibodies.

As used herein, an EGFR inhibitor is an agent that inhibits or reduces the formation of p-Tyr/EGFR (activated EGFR), compared to the formation of p-Tyr/EGFR that would occur in the absence of the EGFR inhibitor. Such inhibitors include small chemical molecules and biologic agents such as monoclonal antibodies.

As used herein, a cell “overexpressing” EGFR (or erbB2) refers to a cell having a significantly increased number of functional EGFR (or erbB2) receptors, compared to the average number of receptors that would be found on a cell of that same type. Overexpression of EGFR and/or erbB2 has been documented in various cancer types, including breast (Verbeek et al., FEBS Letters 425:145 (1998); colon (Gross et al., Cancer Research 51:1451 (1991)); lung (Damstrup et al., Cancer Research 52:3089 (1992), renal cell (Stumm et al, Int. J. Cancer 69:17 (1996), Sargent et al., J. Urology 142: 1364 (1989)) and bladder (Chow et al., Clin. Cancer Res. 7:1957 (2001); Bue et al., Int. J. Cancer, 76:189 (1998); Turkeri et al., Urology 51: 645 (1998)). Overexpression of EGFR and/or erbB2 may be assessed by any suitable method as is known in the art, including but not limited to imaging, gene amplification, number of cell surface receptors present, protein expression, and mRNA expression. See e.g., Piffanelli et al., Breast Cancer Res. Treatment 37:267 (1996).

As used herein, “solid tumor” does not include leukemia or other hematologic cancers.

As used herein, an “epithelial tumor” is one arising from epithelial tissue.

Inhibitors of the tyrosine kinase domains of EGFR or erbB2 used in the present methods should preferentially inhibit phosphorylation of tyrosine residues within the kinase domain, which are the residues implicated in regulating downstream MAPK/Erk and PI3K/AKT pathways. GW572016 is a reversible, dual inhibitor of the tyrosine kinase domains of both EGFR and erbB2.

Non-erbB transactivating factors (such as growth hormone, which is increased in many cancer patients) regulate phosphorylation of tyrosine residues external to the catalytic kinase domain (e.g., Y992, Y1068, Y1148, and Y1173). When conducting immunohistochemistry (IHC) to assess the phosphorylation state of EGFR or erbB2, the use of anti-receptor antibodies that are not domain specific will not distinguish between phosphorylation events in tyrosine residues in the kinase domain and those external to the kinase domain; in this situation the overall phosphorylation state of EGFR and erbB2 may appear unchanged even when key residues within the kinase domain that regulate downstream Erk and AKT pathways may have been inhibited. Accordingly, the use of antibodies that are domain specific is preferred when IHC is utilized in the methods of the present invention.

Biological Markers in Clinical Medicine

The identification of tumor characteristics or biomolecules that can be utilized as surrogate markers to predict the clinical response of an individual patient to a particular treatment (medicine response markers) will be of assistance in clinical practice, to identify those subjects most likely to respond favorably to a given treatment as well as those who are not likely to respond (and who should thus be considered for alternative treatments). Additionally, such markers may be used in clinical trials to identify groups of patients that respond (or do not respond) to a particular therapy, to identify traits and phenotypes common to responders and non-responders.

Anderson et al. (Int. J. Cancer 94:774 (2001) report that the EGFR tyrosine kinase inhibitor ZD1839 (Iressa™) inhibited the proliferation of human cancer cell lines both in vitro and in vivo (animal), and investigated the effects of ZD1839 on activation of EGF-stimulated downstream signals such as ERK Map Kinases. Reductions in tumor growth rates (SKOV3 and MDA-MB-231 xenografts in mice) were reported to coincide with inhibition of constitutive ERK MAP kinase activation. Preincubation of cells with ZD1839 was also reported to block EGF-induced increases in activation of ERK MAP kinases and PKB/Akt in SKOV (human ovarian cancer) cells. No correlation between clinical responseto ZD1839 in humans and ERK MAP kinase activation was reported.

Albanell et al., (Seminars in Oncology, 5 (Supp. 16):56 (2001)) report that administration of the selective EGFR tyrosine kinase inhibitor ZD 1839 to humans in phase I clinical trials resulted in decreased expression of both activated MAPK and Ki-67 in keratinocytes (assessed in biopsies of normal skin taken prior to and following ZD1839 administration; the basal layer of epidermis has high levels of EGF receptor expression). Albanell et al. (J. Clin. Oncol. 20:4292 (2002) ZD1839 reported that serial skin biopsies taken before treatment and at approximately day 28 indicated inhibition of the EGFR signaling pathway.

Albanell et al. (Cancer Research 61:6500 (2001)) reported that in patients treated with a chimeric anti-EGF receptor antibody (Cetuximab; C225), activation of ERK1/2 in skin was lower compared to control (non-patient) skin. Effects on ERK1/2 activation in tumor tissue was not reported.

It has been reported that ERK1 and ERK2 can prime Estrogen Receptor (ER) signalling via phosphorylation of the ER, and that exaggerated ERK1/2 MAPK activity might be capable of driving ER signaling, and thus tumor growth, in the absence of estrogen (phenotypically evidenced as hormone resistance in the tumor) (see e.g., Coutts & Murphy, Cancer Research 58:4071 (1998)). Gee et al. (Int. J. Cancer (Pred. Oncol) 95:247 (2001)) reported that, in a collection of breast cancer tissue samples, pMAPK positive status was found in 83% of ER negative breast cancers; these authors reported that increased ERK1/2 MAPK phosphorylation was associated with earlier relapse on therapy and reduce survival time in both ER negative and ER positive disease.

The present invention correlates the clinical effect of a dual erbB2/EGFR inhibitor in human subjects, with its effects on levels of pERK, pAKT and cyclin D1 in sequential tumor biopsies. The present invention is based on the finding that, in human patients, decreases in particular molecular markers in tumor tissue were correlated with an individual's clinical response to anti-tumor therapy using a dual erbB2/EGFR inhibitor.

The present invention provides a method of screening subjects receiving EGFR inhibitor and/or erbB2 inhibitor, or dual EGFR/erbB2 inhibitor treatment for a solid tumor, to identify those subjects who are most likely to respond favorably to the treatment. Stated another way, the present invention provides a method of screening an individual subject receiving such treatment for a solid tumor, to identify whether the subject is likely to respond favorably to that treatment, as an aid in clinical decision-making.

The methods of the present invention are suitable for use in subjects afflicted with a solid tumor, preferably of epithelial origin, that expresses EGFR or erbB2, and more preferably one that expresses both EGFR and erbB2. In one embodiment of the present invention, the subject is afflicted with a solid tumor of epithelial origin that over-expresses EGFR and/or erbB2.

The methods of the present invention comprise determining the pre-treatment and initial treatment levels of a biological marker in a subject's tumor. Any suitable method of determining the level of specific biological marker may be utilized in the present methods. One such method involves obtaining a biopsy sample of, or cell aspirate from, the subject's tumor and assessing marker levels by any suitable means, as would be apparent to one skilled in the art. The pre-treatment sample may be from tumor tissue that was surgically excised as part of the treatment plan, or may be from a biopsy done solely for determination of marker levels. Tissue must be processed in a manner that allows accurate detection of phosphorylated proteins. E.g., if the tissue sample is paraffin-embedded, it may be fixed in the presence of phosphatase inhibitors and in a neutralized buffered formalin solution.

According to the method of the present invention, the pre-treatment level of a specified marker or markers in the subject's tumor tissue are assessed immediately before the subject begins a course of anti-neoplastic therapeutic treatment. (As used herein, ‘immediately’ before treatment refers to a biologically relevant time frame. Preferably the assessment is done within about three weeks prior to treatment, more preferably within about two weeks, ten days, one week, five days or three days prior to treatment.) After an initial treatment period has passed, the level of the same marker or markers are re-assessed to determine whether the markers in the subject's tumor tissue have increased or decreased. As discussed below, a decrease in pERK, pAKT, and/or cyclinD indicates the subject is more likely to respond favorably to EGFR inhibitor treatment and/or erbB2 inhibitor treatment (or dual EGFR/erbB2 inhibitor treatment), compared to a similar subject with unchanged or increased levels of these markers. Preferably the subject exhibits at least about a 30% decrease in pERK index (calculated as described herein), or a comparable decrease in pERK calculated in a different manner; more preferably the decrease in pERK index (or comparable measure) is at least about 50%, 70%, 80%, or greater. Preferably the subject further exhibits a decrease in pAKT and/or cyclin D1; at least about a 30% decrease in pAKT and/or cyclin D1, more preferably a decrease of at least about 50%, 70%, 80%, or greater.

As is known in the art, clinical use of an antineoplastic agent typically involves repeated administration of the agent to a subject over a set time period, on a pre-established schedule. Therapeutic agents may be administered in any suitable method, including but not limited to intravenously (intermittently or continuously) or orally. For example, a ‘course’ of a certain therapeutic agent may require daily administration of the agent for two weeks; a course of therapy using a different therapeutic agent or for a different tumor type may involve once weekly administration for six weeks. As used herein, a “course” of therapy refers to a therapeutic schedule (dosage, timing of administration, and duration of therapy) that is specific to the therapeutic agent being used and/or the tumor type being treated, and that is accepted in the art as therapeutically effective. Such schedules are developed using pharmacologic and clinical data, as is known in the art. A subject may undergo multiple courses of treatment over time, using the same or different therapeutic agents, depending on whether disease progression occurs.

The present methods are suitable for use in subjects undergoing their first course of antineoplastic treatment, or subjects who have previously received a course of antineoplastic treatment for a tumor.

In the methods of the present invention, the levels of biological markers are assessed pre-treatment, and are re-assessed at some point during treatment (after an initial treatment period). Re-assessment of marker levels preferably occurs at a time when the therapeutic agent has physically reached the site of the tumor for a period sufficient to allow a biological response to the therapeutic agent in the tumor tissue. In one embodiment of the present invention, the initial treatment period is that period of time required for the therapeutic agent to reach steady-state plasma concentratation (or shortly thereafter). Preferably the re-assessment of biological markers occurs shortly after the initial treatment period and prior to the end of a course of therapy, so that therapy may be discontinued in subjects who are not likely to respond. However, re-assessment may also be conducted at or immediately following the end of a course of therapy, to determine if the subject would be suitable for a second course of the same therapy, if required.

The present methods are particularly suited for use with any EGFR, erbB2, or dual EGFR/erbB2 inhibitor, including organic molecules such as GW572016, monoclonal antibodies, or other chemical or biological therapeutic agents.

Any suitable method of detecting specific biological markers may be used in the present methods. One preferred method utilizes immunohistochemistry, a staining method based on immunoenzymatic reactions using monoclonal or polyclonal antibodies to detect cells or specific proteins such as tissue antigens. Typically, immunohistochemistry protocols include detection systems that make the presence of the markers visible (to either the human eye or an automated scanning system), for qualitative or quantitative analyses. Various immunoenzymatic staining methods are known in the art for detecting a protein of interest. For example, immunoenzymatic interactions can be visualized using different enzymes such as peroxidase, alkaline phosphatase, or different chromogens such as DAB, AEC or Fast Red.

The methods of the present invention may be accomplished using any suitable method or system of immunohistochemistry, as will be apparent to one skilled in the art, including automated systems, quantitative IHC, semi-quantitative IHC, and manual methods.

As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems may include automated staining (see, e.g, the Benchmark™ system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Any suitable method of detecting phosphorylated AKT may be used in the present methods, including Western Blotting, immunoprecipitation and Western Blotting, immunohistochemistry, fluorescence in situ hybridization (FISH), and enzyme immunoassays, as are known in the art. Antibodies specific for Ser(473)phospho-AKT are available (see, e.g., Srinivasan et al., Am J Physiol Endocrinol Metab October 2000; 283(4):E784-93).

Any suitable method of detecting phosphorylated ERK1 and ERK2 may be used in the present methods, including Western Blotting, immunoprecipitation and Western Blotting, immunohistochemistry, fluorescence in situ hybridization (FISH), and enzyme immunoassays, as are known in the art. Antibodies that react with p-erk1 and p-erk2 are commercially available (e.g., from Santa Cruz Biotechnology, Santa Cruz, Calif.); see also U.S. Pat. No. 6,001,580).

Any suitable method of detecting or measuring levels of expressed cyclin D1 may be used in the present methods, including Western Blotting, immunoprecipitation and Western Blotting, immunohistochemistry, fluorescence in situ hybridization (FISH), and enzyme immunoassays, as are known in the art. Antibodies that react with cyclin D1 are commercially available (e.g., from Ventana Medical Scientific Instruments (VMSI), Tucson, Ariz.).

The present studies include work in animals with human tumor xenografts. To reduce inter-animal baseline differences in the levels of activated EGFR, Erk1/2 and AKT, in each animal the same tumor implant was biopsied before and after treatment. This approach mimics the clinical setting where each patient serves as his or her own control. In tumor implants, GW572016 inhibited receptor autophosphorylation, as well as downstream p-Erk1/2 and p-AKT. The dual inhibitory nature of GW572016 was demonstrated in BT474 xenografts where GW572016 inhibited activated erbB2 as well as EGFR. The effects of GW572016 on activated erbB2 and EGFR, as well as the activation of downstream intermediaries, correlate with previously reported growth inhibition of both BT474 and HN5 xenografts at the doses of GW572016 administered in the current study (Rusnak et al., Mol. Cancer Therap., 1:85 (2001)).

The inability of EGF to reverse the anti-tumor effects of GW572016 at a molecular level and on the proliferation of cells in vitro is noteworthy, as some tumors may not quantitatively overexpress either erbB2 or EGFR, yet depend upon these receptors for growth and survival signals. Even low levels of EGFR may be activated in the presence of ligands such as EGF, followed by formation of EGFR/erbB2 heterodimers and erbB2 transactivation. GW572016 is a reversible dual inhibitor of both erbB2 and EGFR, and its effects on pathways involved in regulating tumor progression and survival are not reversed in the presence of EGF receptor ligands.

The biological effects of erbB2 and EGFR inhibitors can be studied in tumor cell lines and xenografts. However, obtaining similar data from human patients presents difficulties. Such studies require sequential tumor biopsies prior to and during therapy. The amount of tumor tissue obtained by biopsy is generally limited, and is usually also heterogeneous, with tumor cells interspersed amongst normal cell counterparts, stromal tissue, and fibrotic tissue.

The clinical studies provided herein report on the biological effects of a dual erbB2/EGFR kinase inhibitor (GW572016) in clinical tumor tissue biopsied both pre- and post-treatment. Using sequential tumor biopsies, it was demonstrated that the inhibition of key components of growth and survival pathways (e.g., p-Erk1/2, p-AKT, cyclin D1) can be correlated with the clinical response to treatment.

P-erk1/2

Most mitogenic signals transduced through growth factor receptor activation ultimately converge on a common downstream effector, Erk1/2 MAP kinase (Egan and Weinberg, Nature, 365:781 (1993)). Activated Erk1/2 serves as a transcription factor regulating tumor cell proliferation and survival (Pulverer et al., Nature, 363:83 (1991)). Increased expression of activated Erk1/2 has been demonstrated in a number of human malignancies (Hoshino et al., Oncogene, 18:813 (1999); Albanell et al, Cancer Res., 61: 6500 (2001)), and overexpression of erbB2 in tumor cell lines results in the upregulation of activated Erk1/2 (Janes et al., Oncogene, 9:3601(1994)).

The present data show that GW572016 inhibits baseline Erk1/2 activation in both EGFR and erbB2-dependent tumor lines. Furthermore, although EGF stimulated the expression of activated p-Erk1/2 in BT474 cells (which express relatively low levels of EGFR), GW572016 inhibited this effect of EGF.

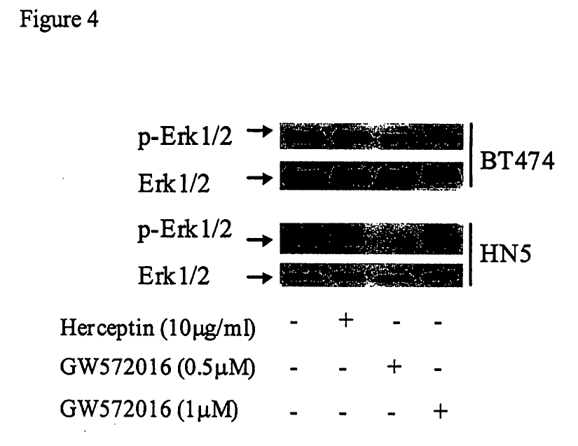

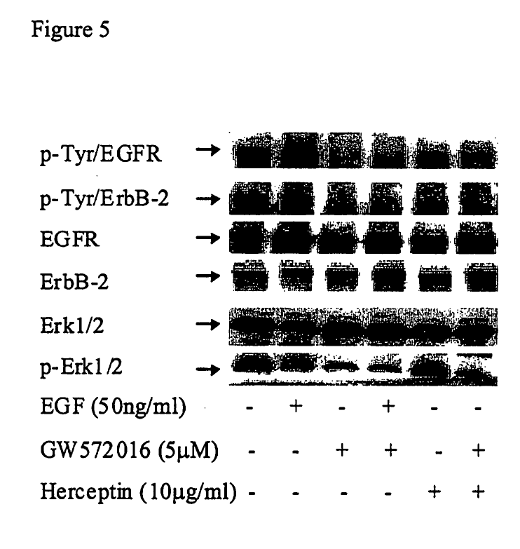

In contrast to GW572016, Herceptin™ did not inhibit Erk1/2 activation in two different erbB2 overexpressing cell lines (see FIG. 4). Herceptin™ did inhibit erbB2 phosphorylation, although less than GW572016 (see FIG. 5). Despite its antiproliferative effects in tumor cell lines, there have been contradictory reports on the effects of Herceptin™ on erbB2 phosphorylation state as well as downstream effectors such as Erk1/2 MAP kinases (Ye et al., Oncogene, 18:731 (1999); Lane et al., Mol. Cell. Biol., 20: 3210 (2000); Scott et al., J. Biol. Chem., 266:14300 (1991)). Differences in cell lines or the time point at which p-Erk1/2 was examined may explain these discrepancies.

Clinical Studies

As reported herein, human cancer patients were treated with GW572016 and the levels of various biomarkers in tumor tissue were compared prior to treatment and after 21 days of treatment. The subjects were re-staged eight weeks following the beginning of treatment. In seven human patients treated with GW572016 who had evaluable day 1 and day 21 tumor biopsies, several demonstrated inhibition of activated Erk1/2 (p-erk) in biopsied tumor tissue. One individual (#361) had metastatic breast cancer (manifesting as subcutaneous nodules) that expressed both EGFR and erbB2; treatment with GW572016 inhibited EGFR p-tyr 32% but did not inhibit erbB2 p-tyr.

In tumor implants, GW572016 inhibited receptor autophosphorylation. The inability to reliably detect significant effects on EGFR/erbB2 p-tyr in clinical subjects whose tumors expressed activated EGFR/erbB2 at baseline may be related to physiologic factors that are relevant in the clinic, but not in laboratory models of cancer. The kinetics of EGFR or erbB receptor turnover and phosphorylation/dephosphorylation is unknown in humans. Additionally, the state of receptor activation is not only regulated by co-expressed erbB3 and erbB4, but also through lateral transactivation of co-expressed G-protein coupled receptors (GPCRs). GPCRs are expressed in many malignancies and their ligands (e.g., angiotensin II) may be expressed in the tumor microenvironment either by paracrine or autocrine mechanisms.

In patient #361, activated Erk1/2 appeared to be completely inhibited upon re-biopsy of one of the metastatic subcutaneous nodules at day 21 of treatment with GW572016. Upon activation and phosphorylation, Erk1/2 translocates from the cytoplasm into the nucleus. Whereas prior to treatment, Erk1/2 protein was almost exclusively expressed in the nucleus of cells from patient #361, at day 21 Erk1/2 protein was almost entirely cytoplasmic, consistent with the inactivation of Erk1/2 in response to treatment with GW572106.

As shown in Table 4, six of eight patients showed at least a 70% decrease in pERK index at day 21 of treatment, compared to pERK index at day 0. (While nine patients were studies, samples from patient #366 exhibited poor quality of staining and were not considered as valid results). Of these six patients, four exhibited either a partial response or stable disease at eight weeks. In contrast, three patients exhibited no decrease or an increase in pERK index; one patient exhibited stable disease while two showed disease progression at eight weeks.

In addition to the ras-MAP/Erk proliferation pathway, erbB receptor heterodimers also activate the PI3K/AKT pathway. Protein kinase B or Akt (PKB/Akt, or AKT) is a serine/threonine kinase, and in mammals comprises three highly homologous members (PKBalpha (Akt1), PKBbeta (Akt2), and PKBgamma (Akt3)). Activated p-AKT is involved in protecting tumor cells from apoptotic stimuli, including cytotoxic agents. In many tumors, constitutive activation of AKT has been implicated as a mechanism of resistance to cytotoxic chemotherapies (Thakkar et al., Oncogene, 20: 6073 (2001); Tenzer et al., Cancer Res., 61: 8203 (2001); Brognard et al., Cancer Res., 61:3986 (2001)). A therapeutic compound that inhibited AKT activation might induce tumor cell apoptosis, either by its own action or by sensitizing tumors to the cytotoxic effects of concurrent chemotherapy.

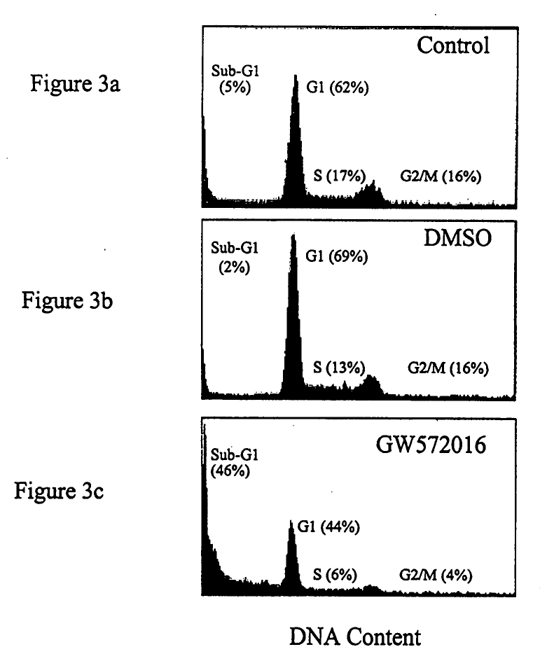

The present data indicate that GW572016 inhibits baseline phosphorylation of AKT in erbB2 (S1) and EGFR (HN5) dependent tumor lines, an effect which was not reversed by the presence of EGF. The ability of GW572016 to inhibit p-AKT was associated in erbB2 (S1) cells with a 23-fold increase in the percentage of S1 cells undergoing apoptosis compared to vehicle treated controls (FIG. 3a-3c). In contrast, apoptosis increased only slightly in HN5 cells (Rusnak et al., Mol. Cancer Therap., 1:85 (2001)). These findings are consistent with recent reports indicating that the PI-3 kinase/AKT pathway appears to be more dependent upon erbB2 signaling than EGFR (Tari and Lopez-Berestein, Int. J. Cancer, 86: 295 (2000)). Since p-AKT inhibition by GW572016 was more pronounced in erbB2 overexpressing cells, induction of apoptosis might in part be dependent upon the degree to which p-AKT is inhibited.

In the clinical studies reported herein, the degree of p-AKT inhibition varied, but correlated with clinical response to therapy. Two patients with metastatic breast cancer refractory to prior therapy with Herceptin™ and multiple chemotherapeutic regimes (subjects 361, 372; see Table 1) demonstrated partial responses while being treated with GW572016, and showed inhibition of p-AKT in response to GW572016 (Table 1). Repeat biopsy from subject #361 showed 33% inhibition of p-AKT after 21 days of treatment with GW572016. Consistent with the pro-apoptotic effects of GW572016 inhibition of p-AKT in tumor cell lines, inhibition of p-AKT in subject #361 coincided with regression of metastatic subcutaneous nodules. The PI3K/AKT pathway has been closely linked to signaling through erbB2/erbB3 heterodimers. Subject #361 over-expressed erbB2 but did not express erbB3. Interestingly, p-Erk1/2 was completely inactivated in the d 21 re-biopsy from subject #361 (Table 1). In this context, activation of erbB2 and EGFR in turn activates Ras kinase, a key intermediary in the Erk proliferation pathway. In addition to regulating proliferation, Ras also regulates the PI3K/AKT pathway by binding to, and activating the p110 subunit of PI3K. While not wishing to be held to a single theory, the present inventors believe that the marked inhibition of p-AKT in subject #361 may be linked to the complete inactivation of the Erk proliferation pathway. A variety of stimuli regulate PI3K/AKT activation including non-erbB receptors such as the platelet-derived growth factor (PDGFR) and insulin-like growth factor receptors (IGFR), both of which are expressed in a variety of tumors and are not targets of GW572016. The expression of these receptors was not determined in the present studies.

In addition to regulating cell survival, AKT is also involved in regulating cell proliferation in part through its modulation of cyclin D protein, which is important in the G1/S phase transition. One of the first events in the initial phase (G1) of the cell cycle is the activation of Cdk4 and/or Cdk6 kinases by the D-type cyclins (D1, D2 and D3). The cyclinD-Cdk4,6 pathway plays a key role in regulating cell growth by integrating multiple mitogenic stimuli. See e.g., Ortega et al., Biochim. et Biophys. Acta 1602:73 (2002). This pathway may be deregulated in human tumors.

In addition to inhibition of p-AKT in subject #361, there was a 90% inhibition of expressed cyclin D1 protein (Table 1). In addition of pAKT in subject #372, there was an 80% decrease in cyclin D1.

Expression of p-AKT, p-Erk1/2, and cyclin D1 was not decreased in two patients (362 and 363) with disease progression eight weeks after treatment with GW572016 (Table 3). In contrast, patient 371 had progressive disease despite inhibition of p-Erk1/2 and cyclin D1 protein (Table 3). However, p-AKT appeared unchanged by therapy, and there was no evidence of therapy-induced tumor cell apoptosis (data not shown). Further investigation revealed that the patient's tumor not only overexpressed EGFR and ErbB2, but also insulin-like growth factor 1 (IGFR-1), which has been implicated in mediating resistance to EGFR inhibitors (Chakravarti et al., Cancer Res. 62:200 (2002)) and Herceptin™ (Lu et al., J. Natl. Cancer Inst. 93:1852 (2001)). In such a situation, one may speculate that activation of the pI3K-AKT survival pathway may have more dependence on IGFR-1 signaling than on EGFR or ErbB2.

Ligand-induced erbB2/EGFR heterodimerization triggers potent proliferative and survival signals. As reported in the cell line and xenograft studies herein, a dual erbB2/EGFR inhibitor (GW572016) inhibited activation of Erk1/2, AKT, and inhibited expression of cyclin D1 (downstream effectors of proliferation and cell survival). Complete inhibition of activated AKT in erbB2 overexpressing cells correlated with a 23-fold increase in apoptosis compared with vehicle controls. EGF, often elevated in cancer patients, did not reverse the inhibitory effects of GW572016. These observations were reproduced in vivo, where GW572016 treatment inhibited activation of EGFR, erbB2, Erk1/2 and AKT in human tumor xenografts. Correlating the changes in pErk1/2, pAKT and cyclin D1 with the clinical response of human subjects treated with GW572016, using serial tumor biopsies, revealed that regression or partial regression of tumors was observed in patients with decreases of pERK, pAKT, and/or cyclin D1.

Biologically Effective Dose

The Maximum Tolerated Dose (MTD) is a current standard method to determine the clinical dose for cytotoxic compounds—the highest dose that does not lead to intolerable side effects is used. However, where the therapeutic agent is targeted to a particular molecule, use of a dose in excess of the available target will potentially add to toxicity without providing any increase in efficacy. GW572016 is a targeted cytostatic agent, inhibiting the EGFR and erbB-2 receptors to cause growth arrest and cellular stasis. For targeted therapeutic agents, use of a maximum Biological Effective Dose (BED) rather than a MTD will provide patients the maximum effect with minimum toxicity.

As used herein, a BED is the dose, or range of doses, of a particular therapeutic compound that produces the optimal biological effect (maximal inhibition of the target). The biological effect upon which the BED is based may differ for compounds having different biological mechanisms of action; the BED dose range will also likely differ among different therapeutic agents and/or among different tumor types. The BED for GW572016 or other EGFR inhibitors (including dual EGFR/erbB2 inhibitors), for example, may be defined as the dose (or range of doses) producing a 75% decrease in EGFR phosphorylation baseline (pre-treatment) levels, or more preferably an 80%, 85% or more decrease in EGFR phosphorylation. Alternatively, the BED for GW572016 or other erbB2 inhibitors (including dual EGFR/erbB2 inhibitors), for example, may be defined as the dose (or range of doses) producing at least a 75% decrease in erbB2 phosphorylation baseline (pre-treatment) levels, or more preferably an 80%, 85% or more decrease in erbB2 phosphorylation.

Data reported herein may be used to estimate the biological effectiveness of GW572016, where effectiveness is defined by inhibition of receptor p-tyrosine phosphorylation. One method of determining a BED is as follows. The percent change from pre- to post-dose biomarker activity level is regressed on dose. An estimate of the dose that produces a pre-selected level of response (e.g., 75% p-tyr inhibition) is calculated by inverse prediction. After this initial BED dose has been estimated, two additional doses are evaluated; these doses are chosen to bracket the dose predicted to yield the desired response. Data from all dose groups are then combined to refine the estimate of the biologically effective dose.

### EXAMPLE 1

Materials and Methods

Materials

The erbB2 overexpressing human breast adenocarcinoma cell line, BT474, was obtained from the American Type Culture Collection (Rockville, Mass., USA). The HB4a cell line was derived from human mammary luminal tissue, and erbB2 transfection of HB4a yielded the cell line HB4a C5.2 (Harris et al., Int. J Cancer., 80:477 (1999)). The S1 cell line was established by sub-cloning HB4a C5.2, and was chosen for further studies as it expressed high levels of phosphorylated erbB2 protein. The EGFR overexpressing LICR-LON-HN5 head and neck carcinoma cell line, HN5, was kindly provided by Helmout Modjtahedi at the Institute of Cancer Research, Surrey, U.K.

EGF was purchased from Sigma Chemical (St. Louis, Mo., USA). Phospho-EGFR and phospho-erbB2 were puchased from Chemicon and NeoMarkers, respectively. Anti-phosphotyrosine antibody was purchased from Sigma Chemical. Anti-EGFR (Ab-12) and anti-c-erbB2 (Ab-11) antibodies were from Neo Markers (Union City, Calif., USA). Additional antibodies to EGFR, erbB2 and Cyclin D1 were obtained from Ventana Medical Scientific Instruments (VMSI, Tucson, Ariz.). Anti-phospho-AKT (Ser 437) and anti-phospho-Erk1/2 were from Cell Signaling Technology, Inc. (Beverly, Mass., USA). Anti-AKT1/2, anti-phospho-Erk1/2, anti-Erk 1 and anti-Erk2 antibodies were also purchased from Santa Cruz Biotechnology, Inc. (Santa Cruz, Calif., USA). Herceptin™ was purchased from Genentech, Inc. (South San Francisco, Calif., USA). SUPERSIGNAL® West Femto Maximum Sensitivity Substrate was from Pierce (Rockford, Ill., USA). Protein G agarose was purchased from Boehringer Mannheim (Germany).

GW572016, N-{3-Chloro-4-[(3-fluorobenzyl)oxy]phenyl}-6-[5-({[2-(methylsulfonyl)ethyl]amino}methyl)-2-furyl]4-quinazolinamine, was synthesized as previously described (Cockerill et al., Bioorganic Med. Chem. Letts., 11:1401 (2001)). GW572016 for cell culture work was dissolved in DMSO.

Cell Cultures

HN5 cells were cultured in DMEM supplemented with high glucose and 10% fetal bovine serum (FBS). HB4a cells grew in RPMI 1640 supplemented with L-glutamine, 10% FBS (Hyclone), 10 μg/ml hydrocortisone, and 5 μ/ml insulin. BT474 cells were cultured under identical conditions to HB4a, but without hydrocortisone. S1 cells were cultured in RPMI 1640 supplemented with L-glutamine, 10% FBS and 50 μg/ml hygromycin. Cell cultures were maintained in a humidified atmosphere of 5% CO₂ at 37° C.

EGF Stimulation Experiments

Cells were seeded at low density in serum free-medium supplemented with 1.5% BSA, and then exposed for 24 h to GW572016 at various concentrations, or 10 μg/ml Herceptin™. Cells were stimulated with 50 ng/ml EGF for 15 minutes, harvested on ice, and then lysed in RIPA buffer (150 mM NaCl, 50 mM Tris-HCl, pH 7.5, 0.25% (w/v) deoxycholate, 1% NP-40, 5 mM sodium orthovanadate, 2 mM sodium fluoride, and a protease inhibitor cocktail).

Cell Cycle Analysis

Cells were harvested and fixed with 70% ethanol in PBS. Cell pellets were then resuspended in 0.5 ml PBS containing propidium iodide (50 μg/ml) and DNase-free RNase (100 μg/ml). Cell cycle analysis was performed using a BD Flow Cytometer (Becton Dickinson, San Jose, Calif., USA).

Immunoprecipitation and Western Blots

Whole cell extracts were prepared by scraping cells off petri dishes, washing the cell pellet twice in phosphate buffered saline (PBS), and then resuspending the pellet in two-packed-cell volumes of RIPA buffer. Protein concentrations were determined using a modification of the Bradford method (Bio-Rad Laboratory). Steady state levels of total erbB2 and EGFR protein, as well activated erbB2 and EGFR were assessed by immunoprecipitation (IP) and Western blot.

For IP Western blots, equivalent amounts of protein were precleared with Protein G Plus/Protein A agarose overnight at 4° C. Precleared lysates were then incubated overnight at 4° C. with specific antibodies. Immune complexes were precipitated with Protein G Plus/Protein A agarose beads, washed in RIPA buffer and then boiled in sample loading buffer. Steady state levels of total Erk1/2 and activated Erk1/2 (p-Erk) as well as total AKT protein and activated AKT (p-AKT) protein were assessed by Western blot. For Western blot, equal amounts of proteins or immunoprecipitated target proteins were resolved by either 7.5% or 4-15% gradient SDS polyacrylamide gel electrophoresis under reducing conditions. Proteins were transferred to Immobilon-P or nitrocellulose membranes. Efficiency and equal loading of proteins was evaluated by Ponceau S staining. Membranes were blocked for 1 hr in TBS (25 mM Tris-HCl, pH 7.4, 150 mM NaCl, 2.7 mM KCl) containing 4% (w/v) lowfat milk or 3% BSA (w/v). Membranes were then probed with specific antibodies recognizing target proteins. Proteins were visualized with the SUPERSIGNAL® West Femto Maximum sensitivity substrate kit (Pierce).

Tumor Xenografts

HN5 cells were grown in DMEM supplemented with 10% fetal bovine serum, sodium pyruvate and L-glutamine at 37° C. in a 95/5% air/CO₂ atmosphere. Cells grown in vitro were harvested in log phase and resuspended in PBS/Matrigel (1:1). Cells (2×10⁶/mouse) in 0.2 ml were injected into the right flank of CD-1 nude mice. Female CD-1 nude mice were acquired from Charles River Laboratories. Mice were maintained in filter-topped cages in an aseptic environment with laminar flow filtered ventilation. Once tumor implants were palpable, mice were administered orally either vehicle (0.5% hydroxypropylmethylcellulose/0.1% Tween 80) alone or five doses of GW572016 at 10, 30, or 100 mg/kg given twice daily at 6 hour intervals. Tumors were biopsied pre-treatment and 4 hours after the last dose. All animal surgery was conducted under aseptic conditions. For the initial biopsy, mice were anesthetized with isofluorane inhalation. The skin over the tumor was disinfected with iodine. A small hemostat was used to tease away the skin from the tumor, and scissors were used to make a 1 cm incision over the tumor. A scalpel and forceps were used to remove approximately 100 mg of tumor. The tumor was then frozen in liquid nitrogen. Wound clips were used to close the incision. The anesthetized mice were kept warm until they recovered mobility, usually less than 1-2 minutes. For the terminal biopsy, mice were euthanized with CO₂ inhalation, and the remainder of the tumor excised. HN5 tumors were placed on dry ice in vials containing cold isopentane, nd stored at −80° C. prior to study. BT474 tumor samples were fixed for 2-3 hours in phosphatase inhibitor consisting of sodium fluoride and sodium pervanadate in 10% neutral buffered formalin. Following fixative treatment, BT474 samples were washed in water and stored in 70% ethanol prior to study. Cell extracts were prepared by homogenization in RIPA buffer at 4° C.

BT474 tumors were maintained by serial passage of fragments into female C.B-17 SCID mice, for up to 10 passages. When tumor implants become palpable, mice were administered either vehicle (0.5% hydroxypropylmethylcellulose/0.1% Tween 80) alone or five doses of GW572016 at 100 mg/kg given twice daily at 12 h intervals by oral gavage. BT474 tumors were removed after the 5^{th} dose of GW572016 after mice were euthanized with CO₂ inhalation. Cell extracts were prepared by homogenization as described for HN5 xenografts.

Immunohistochemistry

Studying the in vivo biological effects of GW572016 using sequential tumor biopsies required an assay that would provide reproducible results with the limited amount of tissue obtained from sequential biopsies, and the heterogeneous nature of those tumor biopsies. Quantitative immunohistochemistry (IHC) was used, which offers an advantage over Western blot analysis in that it provides direct visualization of the effects of GW572016 in tumor cells, which are interspersed amongst surrounding fibrotic tissue, normal cell counterparts, and stroma.

Quantitative Immunohistochemistry (IHC) was performed as previously described (Bacus et al., Analyt. Quant. Cytol. Histol. 19:316-328 (1997). Since phospho-proteins are sensitive to phosphatases activated during tissue procurement, the IHC methodology was refined using tissue from erbB2 (BT474) and EGFR-dependent (HN5) human tumor xenografts. The refined methodology is provided below.

10% Neutral Buffered Formalin Paraffin blocks were sectioned at 4 microns and the sections placed onto coated slides. Sections for p-Erk1/2, p-AKT, p-EGFR, and p-erbB2 were dried in a 60° C. oven for 1 hour. EGFR, erbB2, and cyclin D1 slides were drained, but not dried in the oven.

EGFR, erbB2, and cyclin D1 immunostaining was performed using pre-diluted EGFR, erbB2, and cyclin D1 antibodies on the Ventana Medical Systems Inc. (VMSI) automated “BenchMark” staining module. The Benchmark assigns and recognizes a unique bar-code for each primary antibody, ensuring that the proper protocol and reagents are used for each primary antibody. Protease 1 was used for enzymatic antigen retrieval for EGFR; “Cell Conditioning” 2, mild, employed for erbB2, and “Cell Conditioning” 1, mild for cyclin D1. The VMSI “I-View” detection kit was used as the detection chemistry for all three of the VMSI pre-diluted primary antibodies. After the antibody specific bar-codes are applied, the entire EGFR, erbB2, and cyclin D1 immunostaining, from section drying and deparaffinization to DAB chromogen was completed online on the “BenchMark”.

Phospho-Erk1/2 (1:100) and p-AKT (1:75), were immunostained using a labeled streptavidin peroxidase technique. Slides for p-Erk1/2 and p-AKT immunostaining were deparaffinized and hydrated to water in the usual manner. Slides were subjected to antigen retrieval using 0.1M citrate buffer, pH 6.0 in the “decloaker” (Biocare Corp.) as per the manufacturer's instructions. After antigen retrieval, slides were quenched in 3% hydrogen peroxidetmethanol and blocked in 10% goat senlm/triton X. Phospho-Erk1/2 and p-AKT primary antibodies were then applied and the sections incubated overnight at 4° C. Afterwards, the slides for p-Erk1/2 and p-AKT were placed onto the Autostainer (Dako Corp.) using the LSAB2 kit (Dako) as the detection chemistry. DAB (Dako) was used as the chromogen. The autostainer was programmed to apply both the link and label for 30 minutes. The DAB incubation time was programmed for 5 minutes.

Phospho-EGFR (1:500) and p-erbB2 (1:40) were also immunostained using a similar streptavidin peroxidase labeled technique. Slides for p-EGFR and p-erbB2 were deparaffinzed and hydrated to water in the usual manner. Phospho-EGFR slides were then antigen retrieved with 1 mM EDTA and slides for p-erbB2 with 0.1M citrate buffer, pH 6.0, in the “decloaker”. After antigen retrieval, the p-EGFR and p-erbB2 slides were quenched in 3% hydrogen peroxide/methanol and blocked with 10% goat serum/triton X. The slides were then loaded onto the “Autostainer”. The incubation times for the p-EGFR and p-erbB2 primary antibodies (90 minutes each); the “LSAB2” detection kit link and label (both 30 minutes), and the DAB chromogen (5 minutes) were programmed in; the program started and ran to completion to complete the immunostaining. After immunostaining, all immunomarkers, EGFR, erbB2, p-AKT, p-ERK, p-EGFR, p-erbB2, and cyclin D1 were counterstained manually with 4% ethyl green (Sigma).

Erk1/2 (1:1200), erbB3 (1:10), heregulin (1:25), and TGFα (1:20) were also immunostained using the BenchMark™ with I-VIEW detection chemistry.

To quantify changes in Erk1/2 activation state, a p-Erk index was established for each biopsy. The p-Erk index was the product of the percentage of cells staining positive for p-Erk1/2 in the tissue section and the OD value for p-Erk1/2 immunoreactivity. Investigators preparing and analyzing tissue sections were blinded to both patient tumor type and response to therapy. OD values of ≦10, 10-15 , and ≧15 roughly correlate to 1, 2+ and 3+ in the HercepTest™ (Dakocytomation, Inc., Denmark) standards, respectively.

### EXAMPLE 2

### GW572016 Inhibits erbB2 Tyrosine Phosphorylation and Downstream Activation of Erk1/2

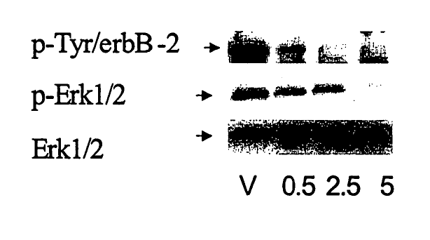

The effects of GW572016 on the activation-state of erbB2 and EGFR, as well as on downstream proliferation and survival pathways, were examined using S1 cells, which overexpress phosphorylated erbB2. S1 cells were established by single cell cloning of Hb4ac5.2 cells, a mammary epithelial line stably transfected with erbB2 (Harris et al., Int. J. Cancer., 80:477 (1999)). GW572016 inhibition of erbB2 tyrosine phosphorylation (i.e. inhibition of the formation of p-Tyr/erbB2) was dose-dependent. Partial inhibition was seen at 500 nM, with complete inhibition at 2.5 μM after 72 h (FIG. 1).

ErbB2 overexpression is associated with the activation of downstream pathways involved in the propagation of proliferative signals such as Erk1/2 MAP kinases (Janes et al., Oncogene, 9:3601(1994)). After 72 h exposure, GW572016 inhibited activated, phosphorylated Erk1/2 (p-Erk) by more than 50% at 500 nM and 2.5 μM, with 100% inhibition at 5 μM compared to vehicle treated controls (FIG. 1). Total steady state Erk protein remained unchanged. A similar dose-dependent relationship was observed in HN5 cells, a squamous cell head and neck carcinoma line that overexpresses EGFR (data not shown).

### EXAMPLE 3

### GW572016 Blocks EGF-Induced Activation of Erk1/2 and AKT in Both erbB2 and EGFR Overexpressing Carcinoma Cells

EGF was recently shown to reverse growth inhibition of OVCA 420 ovarian carcinoma cells treated with combination Herceptin™ and C225 (mAbs targeting erbB2 and EGFR, respectively (Ye et al., Oncogene, 18:731 (1999)). The authors concluded that dual inhibition of EGFR and erbB2 would result in more effective anti-tumor activity.

Since EGF levels have been shown to be elevated in some cancer patients (Grandis et al., J. Natl. Cancer Inst., 90:824 (1998); Albanell et al, Cancer Res., 61: 6500 (2001)), we next examined whether EGF could reverse GW572016 inhibition of activated EGFR, erbB2, and downstream effector molecules.

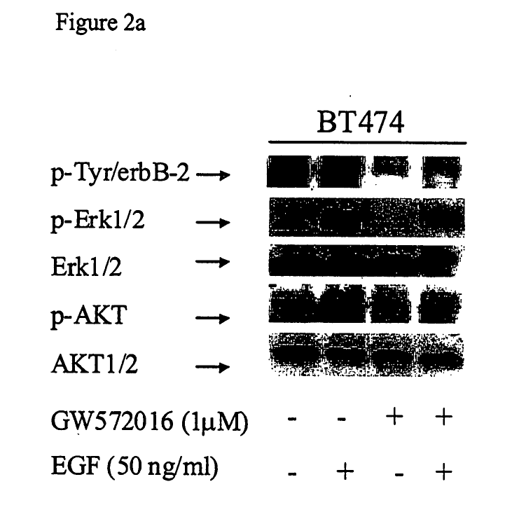

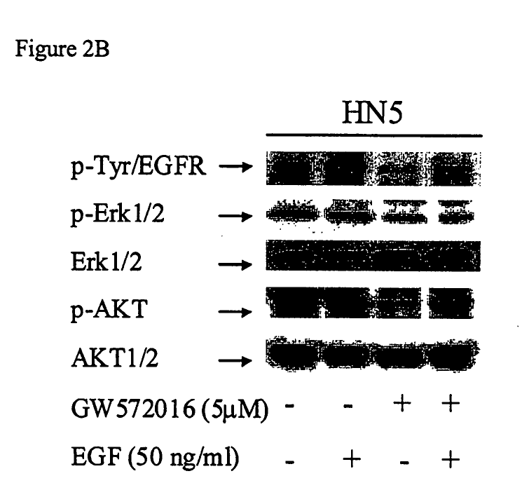

BT474 is an erbB2 overexpressing breast carcinoma line that also expresses EGFR, albeit at lower levels. BT474 cells constitutively express activated erbB2 (p-Tyr/erbB2). BT474 cells were cultured in the presence or absence of GW572016 (1 μM) in serum-free medium for 24 hours. EGF (50 ng/ml) was added to cell cultures as indicated (FIG. 2a) Equal amounts of protein were used to assess activated erbB2 (p-Tyr/erbB2) and Erk1/2, p-Erk1/2, AKT, p-AKT by Western Blot, as described in Example 1. Results are shown in FIG. 2a. EGF stimulation did not significantly increase steady state levels of p-Tyr/erbB2, consistent with this receptor being maximally activated in BT474 cells at baseline (Lane et al., Mol. Cell. Biol., 20: 3210 (2000)). Although EGFR is constitutively expressed at only low levels in BT474 cells, stimulation with EGF increased p-Erk1/2 levels indicating that EGFR signaling was functional. Exposure to 1 μM GW572016 for 24 h inhibited EGF stimulation of p-Erk1/2. GW572016 also inhibited baseline levels of p-Tyr/erbB2, an effect not reversed by EGF.

ErbB2 signaling also activates the PI3K/AKT pathway, which plays an important role in regulating cell survival (Daly R J. Growth Factors, 16:255 (1999)). Constitutive activation of AKT has been implicated in tumor resistance to chemotherapeutic agents (Thakkar et al., Oncogene, 20: 6073 (2001); Tenzer et al., Cancer Res., 61: 8203 (2001); Brognard et al., Cancer Res., 61:3986 (2001)). Stimulation of BT474 cells with EGF increased levels of activated, phosphorylated AKT (p-AKT, Ser 473) approximately 2-fold over baseline (FIG. 2a). In contrast, GW572016 treatment completely inhibited p-AKT. Exogenous EGF did not reverse inhibition.

The effects of GW572016 were examined in HN5 carcinoma cells, which overexpress EGFR (FIG. 2b). HN5 cells were cultured in the presence or absence of GW572016 (5 μM) in serum-free medium for 24 hours. EGF (50ng/ml) was added to cell cultures as indicated (FIG. 2b) Equal amounts of protein were used to assess activated EGFR (p-Tyr/EGFR) and Erk1/2, p-Erk1/2, AKT, p-AKT by Western Blot, as described in Example 1. Results are shown in FIG. 2b. EGFR phosphorylation increased in response to 50 ng/ml EGF. Treating cells with 5 μM GW572016 not only inhibited baseline levels of p-Tyr/EGFR, but also blocked the stimulatory effect of EGF on p-Tyr/EGFR. As in erbB2 overexpressing cells, GW572016 treatment also inhibited downstream p-Erk1/2 in HN5 cells. Simultaneous administration of EGF did not reverse these inhibitory effects. Although GW572016 treatment inhibited p-AKT in HN5 cells, the effect was smaller than in erbB2-overexpressing tumor cells.

### EXAMPLE 4

### ErbB2 Overexpressing Cells Undergo Apoptosis in Response to GW572016

The effects of GW572016 on cell survival were assessed in exponentially growing S1 cells (erbB2 overexpressing cells derived from human mammary tissue). S1 cells in exponential log growth phase were treated with GW572016 (5 μM), vehicle (0.1% DMSO), or were untreated controls. After 72 h, cell cycle analysis was performed using propidium iodide staining and flow cytometry as described in Materials and methods.

Results are shown in FIGS. 3a-3c. The sub-G1 peak represents the apoptotic fraction, and comprised 2% of vehicle-treated (control) S1 cells. The percentage of apoptotic cells increased 23-fold to 46% after 72 h exposure to GW572016, with a concomitant reduction in the percentage of cells in S phase and G2/M. A similar result was observed in BT474 cells (Rusnak et al., Mol. Cancer Therap., 1:85 (2001)). Although growth arrest was seen in HN5 cells treated with GW572016, significant apoptosis was not seen (data not shown), consistent with previous observations (Rusnak et al., Mol. Cancer Therap., 1:85 (2001)).

### EXAMPLE 5

The Effects of GW572016 on Erk1/2 Activation State Differ from that of Herceptin™

Herceptin™, a humanized anti-erbB2 mAb, exhibits activity in the clinic against breast cancers that either overexpress erbB2 protein or demonstrate erbB2 gene amplification (Cobleigh et al, J. Clin. Oncol., 17:2639 (1999)). However, the exact mechanism by which Herceptin™ exerts its anti-tumor activity is unclear. We compared the effects of GW572016 with Herceptin™ on p-Erk1/2 in both BT474 (erbB2 overexpressing) and HN5 (EGFR overexpressing) cells, using treatment conditions for Herceptin™ (10 μg/ml) previously shown to inhibit the growth of erbB2 overexpressing cells (Lane et al., Mol. Cell. Biol., 20: 3210 (2000)). Exponentially growing BT474 (erbB2 overexpressing) and HN5 (EGFR over-expressing) cells were cultured with either GW572016 (0.5 μM or 1 μM) or Herceptin™ (10 μg/ml) for 72 hours. Cell lysates were prepared and total Erk1/2 and activated Erk1/2 (p-Erk1/2) were assessed by Western blot.

Results are shown in FIG. 4. At 72 hours, Herceptin™ had very little effect on p-Erk1/2 levels compared with untreated controls in either cell line, while GW572016 at 500 nM or 1 μM inhibited p-Erk1/2 in both BT474 and HN5 cells. Neither Herceptin™ nor GW572016 reduced total Erk1/2 steady state protein levels.

### EXAMPLE 6

### GW572016 and Herceptin™ Elicit Differential Effects on the Activation State of erbB2, EGFR and Downstream Erk 1/2 in Cells Expressing Low Levels of erbB2 and EGFR

Hb4a is a mammary epithelial line that expresses low levels of both erbB2 and EGFR (Harris et al., Int. J. Cancer., 80:477 (1999)). Exponentially growing Hb4a cells were treated with either 5 μM GW572016 or Herceptin™ (10 μ/ml) for 72 h and stimulated with EGF (50 ng/ml) for 15 minutes as described in Materials and Methods. Steady state levels of activated erbB2 and EGFR (p-Tyr/ErbB2 and p-Tyr/EGFR); total erbB2 and EGFR; activated Erk1/2 (p-Erk1/2) and total Erk1/2 were assessed by either IP Western or Western blot.

As shown in FIG. 5, steady state p-Tyr/EGFR levels increased in response to EGF stimulation, and indicated the integrity of the EGFR pathway in these cells. GW572016 not only reduced baseline p-Tyr/EGFR levels in Hb4a cells but also blocked the stimulatory effects of EGF on EGFR tyrosine phosphorylation. Similarly, GW572016 reduced the baseline amount of p-Tyr/erbB2 and p-Erk, effects not reversed by EGF.

As also shown in FIG. 5, after 72 h exposure to Herceptin™, there was relatively little change in baseline levels of p-Tyr/erbB2 or p-Erk levels, while total erbB2 steady state protein was reduced. Concurrent treatment with GW572016 and Herceptin™ did not reduce levels of p-Tyr/erbB2 or p-Erk below those observed following treatment with GW572016 alone.

Exponentially growing Hb4a cells were cultured in 35 mm petri dishes with serum-free medium containing 1.5% BSA. Treatment conditions included: DMSO (final concentration of 0.1%) as the vehicle control; EGF (50 ng/ml); GW572016 (2.5 μM); concurrent GW572016 (2.5 μM)+EGF (50 ng/ml). Viable cells were counted after 72 h using trypan blue exclusion. Data from three independent experiments indicated that EGF stimulated Hb4a cell growth by 20% over vehicle treated (DMSO) controls, while treatment with GW572016 (2.5 μM) inhibited cell growth 50% compared with vehicle treated controls (data not shown). EGF did not reverse GW572016 induced growth inhibition.

### EXAMPLE 7

### In vivo Inhibitory Effects of GW572016 on Receptor p-Tyr Expression and Downstream Signaling Components—Tumor Xenografts

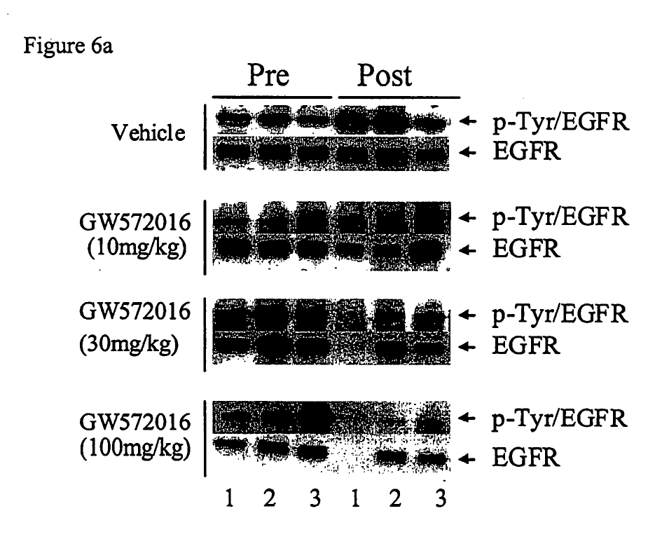

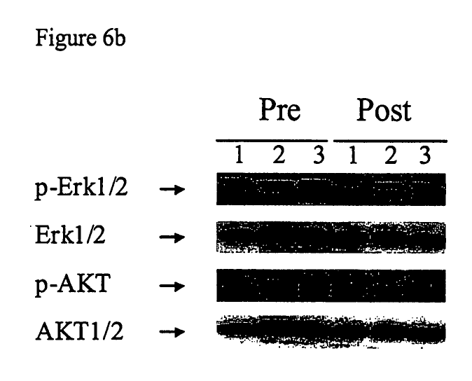

The effects of GW572016 on the activation of EGFR and downstream pathways were examined in the HN5 human tumor xenograft model. HN5 tumor xenografts were established subcutaneously in CD-1 nude mice as described in Materials and methods. When tumors were palpable, treatment with GW572016 was initiated at the indicated doses; controls were treated with vehicle alone. Vehicle or GW572016 was administered by oral gavage twice daily at a six hourly interval, for five doses. To simulate the clinical setting where each patient serves as his or her own control, each animal was used as its own control, by taking biopsies from the same tumor implant before (pre) and after (post) the final treatment dose of GW572016; each treatment cohort comprised three animals (indicated as 1, 2, and 3 in FIG. 6). Activated receptor (p-Tyr/EGFR) was assessed by IP Western blot and total EGFR steady state protein (EGFR) by Western blot.

As shown in FIG. 6a, GW572016 treatment resulted in a dose-response effect, with very little inhibition of p-Tyr/EGFR at 10 mg/kg, increasing at 30 and 100 mg/kg. One of the post-therapy biopsies was not evaluable at each of the two higher doses, as the samples contained inadequate EGFR protein.

The effects of GW572016 treatment on Erk and AKT were also examined. Total Erk1/2, total AKT, activated Erk1/2 (p-Erk1/2), and activated AKT (p-AKT) were assessed by Western blot loading equal amounts of protein from tumor biopsies. There was little effect in animals treated with vehicle alone or administered 10 mg/kg GW572016 (data not shown). However, at 30 mg/kg/dose, GW572016 inhibited p-Erk1/2 and p-AKT in tumors without affecting total steady state protein levels of either molecule (FIG. 6b). Treatment at 100 mg/kg/dose showed similar inhibition of p-Erk and p-AKT (data not shown).

To highlight the dual inhibitory nature of GW572016, the effects of GW572016 on the activation state of erbB2 and Erk1/2 in BT474 (erbB2 overexpressing) xenografts were examined. BT474 tumor xenografts (subcutaneous) were established as described in Materials and Methods. When tumors were palpable, GW572016 (100 mg/kg) was administered by oral gavage twice daily at six hourly intervals, for five doses. Controls were treated with vehicle alone. In contrast to HN5, BT474 tumor implants were not amenable to re-biopsy; the tumor implant was removed after the 5^{th} dose of GW572016. Each treatment cohort comprised three animals: vehicle (lanes 1, 2, and 3) and GW572016 (lanes 4, 5 and 6). Activated receptor (p-Tyr/ErbB-2) was assessed by IP Western blot and total ErbB-2 steady state protein (ErbB-2), total Erk1/2 and activated Erk1/2 (p-Erk1/2) were assessed by Western blot loading equal amounts of protein from tumor biopsies.

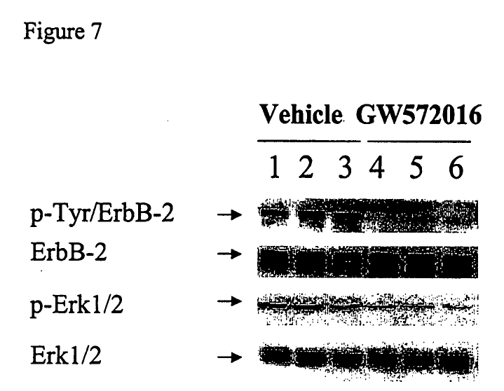

Both p-Tyr/erbB2 and p-Erk1/2 were inhibited by GW572016 without effects on total erbB2 or Erk1/2 steady state protein levels (FIG. 7).

### EXAMPLE 8

### GW572016 Inhibits Activated EGFR, erbB2 and downstream Proliferation Signaling Pathways in Tumor Xenografts—Assessed by Quantitative Immunohistochemistry

As reported in the examples above, inhibition of erbB2 or EGFR tyrosine autophosphorylation by GW572016 led to the inactivation of Erk1/2 and AKT in tumor cell lines and xenografts, although AKT was more potently inhibited in erbB2 driven tumor lines (see example 3, above). These data were obtained using Western blot analysis. However, Western blot techniques are often not practical in clinical studies, where the amount of tissue obtained from sequential tumor biopsies is limited and the content of biopsies may be heterogeneous. To evaluate sequential tumor biopsies, we utilized quantitative immunohistochemistry (IHC), which (i) enables confirmation of the presence of tumor cells within biopsies, (2) provides direct visualization of the effects of a test compound on tumor cells, which are interspersed amongst surrounding fibrotic tissue, normal cell counterparts, and stroma, and (3) biological parameters can be assessed using the limited amount of tissue available from sequential biopsies.

The effect of GW572016 on total erbB2, EGFR, and the activated tyrosine phosphorylated forms of the erbB2 and EGFR receptors was examined using IHC in erbB2 (BT474) and EGFR-dependent (HN5) human tumor xenografts. Administration of 200 mg/kg GW572016 by oral gavage once daily in tumor-bearing mice led to the inhibition of activated EGFR and erbB2 in a dose-dependent manner in both HN5 and BT474 xenografts, whereas neither EGFR nor erbB2 total protein was affected (data not shown).

Inhibition of erbB2/EGFR p-tyr in turn led to the inhibition of downstream activated, phospho-Erk1/2. Whereas p-Erk1/2 levels were reduced in response to GW572016, total Erk1/2 protein was unaffected (data not shown).

### EXAMPLE 9

Clinical Trial of GW572016—Protocol

An open-label study of multiple doses of GW572016 was conducted to examine the inhibition of EGFR/erbB-2 phosphorylation in patients with solid tumors. This study looked at the effect of GW572016 on the expression of the activated, tyrosine phosphorylated forms of erbB-2 and/or EGFR, and other molecules associated with tumor cell proliferation and survival pathways (e.g., ERK1/2, AKT, cyclin D1).

Patients (males and females) 18 years or older, with histologically confirmed epithelial tumors were eligible for treatment with GW572016 if their tumors over-expressed either EGFR or erbB2 (or both), or in the case of erbB2, exhibited gene amplification. Subjects entered in this study had previously failed, or were not eligible for, standard antineoplastic treatment. Patients received GW572016 at fixed doses of 500, 650, 900, 1200 or 1600 mg/day administered orally on a once a day schedule. Patients were randomized to receive one of the five doses of GW572016 (provided as tablets of GW572016 ditosylate salt). All subjects provided written informed consent. Tumor biopsies were obtained immediately prior to initiation of therapy (d 0) and again 21 days (d 21) after starting therapy. Day 21 was chosen based on evidence that steady state plasma concentrations of GW572016 were achieved by that time.

Prior to treatment with GW572016, the EGFR and/or erbB-2 status were determined for each patient from archived tumor tissue (collected at time of diagnosis) or, if archived tissue was unavailable, from a current biopsy. Biopsies of tumors for determination of erbB-2 and/or EGFR phosphorylation was done prior to the first dose of GW572016 (Day 0). Only patients with tumors that over-expressed total EGFR by immunohistochemistry (IHC) and/or overexpressed total erbB-2 by IHC or fluorescence in situ hybridization (FISH), or expressed activated EGFR and/or erbB-2 as determined by semi-quantitative IHC were studied. In addition, all patients had tumors that were readily accessible to biopsy. Tumors were also analyzed for cell proliferation molecules (e.g., ERK1/2, p-ERK1/2, AKT, p-AKT and cyclin D1)

On Day 21 of dosing, a second tumor biopsy was obtained within 12 hours and as close to 4 hours as possible after the 21^{st} GW572016 dose. Day 21 biopsy samples were evaluated, including evaluation for cell proliferation molecules (e.g.,p-ERK, p-AKT, cyclin D1). Data are provided in Tables 14.

In Tables 1-3, the Optical Density (OD) scores were obtained using a computerized system (VMSI BenchMark™) that scanned the slides and applied an OD number representing the intensity of staining. The computer was initially ‘trained’ using a single trained human observer's scoring of slides; use of the computerized system thus reduces inter-operater variability of scoring.

“EGFR” refers to total EGFR as measured by immunohistochemistry, and reported as Optical Density (OD);

“erbB2” refers to total erbB2 as measured by immunohistochemistry and reported in OD;

“erbB3” (HER3) refers to total erbB3 as measured by immunohistochemistry and reported in OD;

“pERK index” is calculated by multiplying the percentage of cells staining positive for p-Erk and the optical density (OD) score, ×100;

“Cyclin D1” refers to total cyclin D1 present as measured by immunohistochemistry and reported by OD;

“pAKT” refers to phosphorylated AKT as measured by immunohistochemistry and reported in OD;

“TGFα” refers to Transforming Growth Factor alpha as measured by immunohistochemistry and reported in OD;

“Heregulin”, a ligand that stimulates erbB3 (HER3) and HER4, was measured by immunohistochemistry and reported in OD.

Subjects had a disease assessment completed within 28 days prior to initial dosing with GW572016; assessment was based on RECIST (Response Evaluation Criteria In Solid Tumors; see Therasse et al., New Guidelines to Evaluate the Response to Treatment in Solid Tumors, J. Natl. Cancer Inst., 92(3):205 (2000)). Re-assessment (“re-staging”) using RECIST criteria was conducted at eight weeks after the initiation of GW572016 therapy. Subjects were thereafter allowed to continue GW572016 therapy with subsequent re-stagings as appropriate.

### EXAMPLE 10

### Effects of GW572016 in Clinical Tumor Biopsies and Correlation with Clinical Response

The biological effects of GW572016 were assessed in the subjects discussed in Example 9, above, using sequential tumor biopsies. Tables 1-4 show the effects of GW572016 on the first nine patients, however, samples from patient #366 exhibited aberrant staining (poor quality of staining) and were not considered as valid results.

In Tables 1-3, an OD value less than or equal to 10 roughly corresponds to HercepTest™ (Dakocytomation, Inc., Denmark) standard 1+; an OD value of 10-15 roughly corresponds to HercepTest™ standard 2+; and an OD value of 15 or more roughly corresponds to HercepTest™ standard 3+. (The HercepTest™ is an immunohistochemical staining procedure used to identify Her2 overexpression, and is clinically useful in identifying patients who may be suitable for treatment with Herceptin™ (Genentech, Inc., South San Francisco, Calif.)).

The results from patient #361 illustrate several points. This individual had metastatic breast cancer, previously treated with a variety of chemotherapeutic agents, both with and without Herceptin™. Despite these therapeutic interventions, her metastatic disease, manifest by painful subcutaneous nodules, progressed. She was randomized to receive 1200 mg/day of GW572016. A baseline (d 1) biopsy from one of her subcutaneous nodules showed tumor over-expression of EGFR and erbB2 receptors, the latter more pronounced than the former (data not shown). Both receptors were activated at baseline (data not shown). Consistent with the preclinical data, treatment with GW572016 had no effect on total erbB2 or EGFR protein. In contrast, EGFR p-tyr was inhibited 32% at Day 21 compared with baseline (data not shown). Interestingly, erbB2 p-tyr had not decreased at Day 21 (data not shown). However, at the time of her Day 21 biopsy, almost all of her metastatic subcutaneous nodules had completely regressed. Although the GW572016 did not greatly decrease either erbB2 or EGFR p-tyr levels, it reduced tumor levels of pErk1/2, pAKT and cyclin D1. Table 1.

Increased expression of pErk1/2 has been demonstrated in a number of malignancies, and is correlated with metastatic disease in breast cancer. Over-expression of erbB2 in cell lines increases expression of activated Erk1/2. To quantitatively assess the effects of GW572016 on Erk1/2 activation-state, a phospho-Erk (p-Erk) index for each biopsy was calculated as the product of the percentage of cells staining positive for p-Erk multiplied by the intensity of the staining (the optical density (OD) score). Subject #361 had an extremely high baseline p-Erk index of 4015 (Table 1); at Day 21 the pErk index was 0. (Table 1).

Upon activation, p-Erk1/2 relocates to the nucleus where it regulates transcription of a variety of genes involved in tumor growth, adhesion, and angiogenesis. Consistent with the high levels of activated Erk1/2 prior to therapy, baseline staining of total Erk1/2 from patient #361 Day I tumor was exclusively intra-nuclear (data not shown). In contrast, total Erk1/2 was almost entirely cytoplasmic at Day 21 (data not shown) consistent with the apparent inactivation of Erk1/2 by GW572016.

The PI3K/AKT pathway plays an important role in protecting tumor cells against apoptosis. Inhibition of p-AKT levels in GW572016-treated tumor cell lines, especially erbB2 over-expressing tumor lines, was associated with the induction of apoptosis. As shown in Tables 1-3, GW572016 modulated levels of activated AKT (p-AKT) levels in tumors to varying degrees. Patient #361, whose metastatic breast cancer had a marked clinical response to GW572016 also demonstrated inhibition of pAKT in response to GW572016 at Day 21.

Cyclin D1 plays a key role in regulating cell cycle progression, and is a key cell cycle regulator involved in GI to S phase transitions. Deregulation of cyclin D has been implicated in the pathogenesis of breast cancer, particularly those tumors overexpressing erbB2. Not only did GW572016 inhibit p-Erk1/2 and p-AKT in Day 21 tumor biopsies from patient #361, it also reduced cyclin D1 protein expression 90% at d21.

Day 21 tumor biopsy from patient #364 demonstrated a >90% decrease in p-Erk1/2 in response to GW572016 (Table 1). This patient received 1600 mg/day GW572016 for refractory metastatic head and neck cancer. In addition, cyclin D1 expression was reduced 50% after 21 days of therapy (Table 1). However, in contrast to patient #361, her p-AKT was only reduced 16%. Interestingly, at the time of her Day 21 biopsy, the metastatic lymph node that was sequentially biopsied had reduced in size, but the response was less pronounced than patient #361.

Other patients when restaged at Week 8 (e.g., #362, 363) were found to have progression of their disease clinically, which was associated with increased p-Erk index, and increases in cyclin D1, and p-AKT (Table 3).

After restaging at Week 8, patients were allowed to continue therapy with GW572016, with restaging every month thereafter. Some patients' disease did progress after the end of the eight week study period.

TABLE 1
Patients Achieving Partial Response at 8 weeks:

| Patient, | | | | | | | | |
|---|---|---|---|---|---|---|---|---|
| Tumor Type, | EGFR | ErbB2 | p-ERK | p-AKT | cyclin D1 | ErbB3 | TGF-α | Heregulin |
| Dose (mg/d) | OD | OD | index | OD | OD | OD | OD | OD |
| 361 - | | | | | | | | |
| Breast - | | | | | | | | |
| 1200 | | | | | | | | |
| Day 0 | 20 | 43 | 4015 | 36 | 31 | 0 | 35 | 20 |
| Day 21 | | | 0 | 24 | 3 | | | |
| 372 - | | | | | | | | |
| Breast - | | | | | | | | |
| 1200 | | | | | | | | |
| Day 0 | 7 | 50 | 378 | 48 | 20 | 60 | 38 | 10 |
| Day 21 | | | 10 | 30 | 4 | | | |

TABLE 2
Patients with Stable Disease at 8 weeks:

| Patient, | | | | | | | | |
|---|---|---|---|---|---|---|---|---|
| Tumor type, | EGFR | ErbB2 | p-ERK | p-AKT | cyclin D1 | ErbB3 | TGF-α | Heregulin |
| Dose | OD | OD | index | OD | OD | OD | OD | OD |
| 364 - | | | | | | | | |
| Head & Neck - | | | | | | | | |
| 1600 | | | | | | | | |
| day 0 | 23 | 11 | 1634 | 51 | 66 | 4 | 59 | 15 |
| day 21 | | | 100 | 43 | 33 | | | |
| 369 - | | | | | | | | |
| Head & Neck - | | | | | | | | |
| 1200 | | | | | | | | |
| day 0 | 26 | 25 | 0 | 24 | 39 | 0 | 49 | 7 |
| day 21 | | | 0 | 7 | 22 | | | |
| 367 - | | | | | | | | |
| Adenocarcinoma, | | | | | | | | |
| unknown primary - | | | | | | | | |
| 650 | | | | | | | | |
| day 0 | 17 | 3 | 230 | 35 | 46 | 0 | 16 | 0 |
| day 21 | | | 0 | 25 | 39 | | | |
| 366 - | | | | | | | | |
| Ovarian - | | | | | | | | |
| 900 | | | | | | | | |
| day 0 | 8 | 2 | 110 | 22 | 0 | 2 | 16 | 0 |
| day 21 | | | 25 | 47 | | | | |

TABLE 3
Patients with Progressive Disease at 8 weeks:

| Patient, | | | | | | | | |
|---|---|---|---|---|---|---|---|---|
| Tumor type, | EGFR | ErbB2 | p-ERK | p-AKT | cyclin D1 | ErbB3 | TGF-α | Heregulin |
| Dose | OD | OD | index | OD | OD | OD | OD | OD |
| 362 - | | | | | | | | |
| Adenocarcinoma, | | | | | | | | |
| Unknown primary | | | | | | | | |
| 900 | | | | | | | | |
| day 0 | 42 | 10 | 576 | 61 | 26 | 0 | 17 | 0 |
| day 21 | | | 1260 | 81 | 37 | | | |
| 363 - | | | | | | | | |
| Sarcoma | | | | | | | | |
| 500 | | | | | | | | |
| day 0 | 10 | 0 | 20 | 19 | 1 | 0 | 13 | 0 |
| day 21 | | | 336 | 32 | 12 | | | |
| 371 - | | | | | | | | |
| Breast | | | | | | | | |
| 900 | | | | | | | | |
| day 0 | 14 | 44 | 1081 | 36 | 42 | 57 | 49 | 7 |
| day 21 | | | 0 | 33 | 10 | | | |

OD measurements obtained using quantitative immunohistochemistry with Ventana Benchmark ™ system.

TABLE 4
Summary of effects of % inhibition of pERK (assessed
by pERK index) for initial nine patients:

| Percentage | | | |
|---|---|---|---|
| decrease in | Number of patients | Response at Eight | |
| pERK index | (Total N = 8)* | Weeks | Tumor type |
| >70% | 6 (66.6%) | Partial Response: 2/5 | Breast (361, 371, 372); |
| | | Stable Disease: 3/5 | Head and Neck (364); |
| | | Progression: 1/5 | Adenocarcinoma, unknown |
| | | | primary (367); |
| No decrease or | 3 (33.3%) | Partial Response: 0/3 | Head and Neck (369); |
| increase | | Stable Disease: 1/3 | Adenocarcinoma, unknown |
| | | Progression: 2/3 | primary (362) |
| | | | Sarcoma (363); |

*samples from patient #366 exhibited poor quality of staining and were not included in Table 4.

Of the five patients with at least a 70% decrease in pERK index, four (80%) had a partial response or stable disease at eight weeks; one showed progression of disease at eight weeks. Of the three patients with no decrease or an increase in pERK index, one (33%) had stable disease at eight weeks, the other two (66%) showed progression of disease.

That which is claimed is:

1. In a human subject in need of treatment with a therapeutic compound for an EGFR-expressing solid tumor, a method to assess whether the subject is likely to exhibit a favorable clinical response to said treatment, comprising:
(a) determining the pretreatment level of pERK in said tumor;
(b) administering a therapeutically effective amount of an agent selected from an EGFR inhibitor, an erbB2 inhibitor, and a dual EGFR/erbB2 inhibitor; and
(c)determining the level of pERK in said tumor after an initial period of treatment with said therapeutic agent,
where a decrease in the pERK level indicates said subject is more likely to exhibit a favorable clinical response to treatment with said therapeutic agent, compared to a subject with no change or an increase in pERK levels.

2. A method according to claim 1 where said initial period of treatment is the time required to achieve a steady-state plasma concentration of said therapeutic compound.

3. A method according to claim 1 where p-erk levels are assessed by immunohistochemical methods.

4. A method according to claim 1 where said p-erk levels are assessed by comparing the distribution of total erk between nucleus and cytoplasmic compartments of the tumor cell.

5. A method according to claim 1 where said tumor also expresses erbB2.

6. A method according to claim 1 where said tumor over-expresses EGFR or erbB2.

7. A method according to claim 1 where said solid tumor is an epithelial tumor.

8. A method according to claim 1 where said tumor is selected from breast, ovarian, colon, head and neck, bladder, renal cell and lung tumors.

9. A method according to claim 1 where said therapeutic agent is a dual EGFR/erbB2 inhibitor.

10. A method according to claim 1 where said therapeutic agent is GW572016.

11. A method according to claim 1 where said therapeutic agent is GW572016 and said initial treatment period is from about 14 days to about 28 days.

12. A method according to claim 1, further comprising determining the level of pAKT in said tumor pre-treatment and after the initial period of treatment.

13. A method according to claim 1, further comprising determining the level of cyclin D1 in said tumor pre-treatment and after the initial period of treatment.

14. In a human subject in need of treatment with a therapeutic compound for an erbB2-expressing solid tumor, a method to assess whether the subject is likely to exhibit a favorable clinical response to said treatment, comprising:
(a) determining the pre-treatment level of pERK in said tumor;
(b) administering a therapeutically effective amount of an agent selected from an EGFR inhibitor, an erbB2 inhibitor, and a dual EGFR/erbB2 inhibitor, and
(c) determining the level of pERK in said tumor after an initial period of treatment with said therapeutic agent,
where a decrease in the pERK level indicates said subject is more likely to exhibit a favorable clinical response to treatment with said therapeutic agent, compared to a subject with no change or an increase in pERK levels.

15. A method according to claim 1 where said initial period of treatment is the time required to achieve a steady-state plasma concentration of said therapeutic compound.

16. A method according to claim 1 where p-erk levels are assessed by immunohistochemical methods.

17. A method according to claim 1 where said p-erk levels are assessed by comparing the distribution of total erk between nucleus and cytoplasmic compartments of the tumor cell.

18. A method according to claim 1 where said tumor also expresses EGFR.

19. A method according to claim 1 where said tumor over-expresses EGFR or erbB2.

20. A method according to claim 1 where said solid tumor is an epithelial tumor.

21. A method according to claim 1 where said tumor is selected from breast, ovarian, colon, head and neck, bladder, renal cell and lung tumors.

22. A method according to claim 1 where said therapeutic agent is a dual EGFR/erbB2 inhibitor.

23. A method according to claim 1 where said therapeutic agent is GW572016.

24. A method according to claim 1 where said therapeutic agent is GW572016 and said initial treatment period is from about 14 days to about 28 days.

25. A method according to claim 1, further comprising determining the level of pAKT in said tumor pre-treatment and after the initial period of treatment.

26. A method according to claim 1, further comprising determining the level of cyclin D1 in said tumor pre-treatment and after the initial period of treatment.
